(19) United States
(12) Patent Application Publication
(10) Pub. No.: US 2009/0117643 A1
(43) Pub. Date: May 7, 2009

(54) Tumor-targeting gene-virus zd55-il 24,its construction method and application thereof

(76) Inventors: Xinyaun Liu, Shanghai (CN); Lili Zhao, Shanghai (CN); Jinfa Gu, Shanghai (CN); Lanying Sun, Shanghai (CN)

Correspondence Address: ZHEN ZHENG LU, 1730 HUNTINGTON DRIVE #304, DUARTE, CA, 91010, US

(21) Appl. No.: 11/920,951

(22) PCT Filed: May 22, 2006
(86) PCT No.: PCT/CN2006/001061

§ 371 Date: Nov. 10, 2008

(30) Foreign Application Priority Data
May 25, 2005 (CN) 200510026151.5

(51) Int. Cl. C12N 7/00

(52) U.S. Cl. 435/235.1

(57) ABSTRACT

This present invention disclosed a tumor-targeting gene-virus ZD55-IL-24, which is a recombinant Ad5 with a deletion of E1B 55K da gene and carries the anti-tumor gene IL-24. This present invention also disclosed its construction method and its application in cancer gene-therapy. The tumor-targeting gene-virus ZD55-IL-24 of this invention can be used in the therapy of many kinds of tumors, so it can be used in developing the new effective tumor therapy medicine.

### BACKGROUND OF THE PRESENT INVENTION

1. Field of Invention

The present invention belongs to the field of gene therapy, and in particular, relates to a novel tumor-targeting gene-virus ZD55-IL-24 that shows tumor-specific proliferation and foreign gene expression, its construction method and application thereof.

2. Description of Related Arts

The gene therapy is a high bio-technique to deliver therapeutic genes into patients, which has developed in the recent 10 years. More than 60% of all gene therapy protocols are for cancer gene therapy. Gene therapy is deemed as a hope for mankind finally to conquer the tumor. Vectors for gene therapy are divided into two types: viral vectors and non-viral vectors. Viral vectors include adenovirus, adeno-associated virus (AAV), retrovirus, lentivirus and herpes virus. Viral vectors have advantages of high transduction efficiency and longer lifetime of foreign gene expression, but have dangers for its strong immunogenicity. Non-viral vectors include naked DNA or capsulated DNA with liposome or other materials. Non-viral vectors have good security for its low immunogenicity, but have disadvantages of low transduction efficiency, poor gene stability and short lifetime of foreign gene expression. At present viral vectors are most widely used. Viral vectors are divided into two types. One type is able to integrate into the chromosome, such as the retrovirus, adeno-associated virus (AAV) and lentivirus. The other type of viral vector, such as the adenovirus, EB virus and HSV, is unable to integrate into the chromosome and stays outside the DNA genome. Among viral vectors, the adenovirus (ad) vectors are most widely used. Ad includes six kinds: A, B, C, D, E and F and 49 types of blood serums. Ad2 and ad5 of C kind are most widely used. The wild-type adenoviruses are DNA viruses with a double-stranded linear genome of 36 kb. The genome is divided into early functional transcription regions (E) and late functional transcription regions (L). The first generation Ad vectors are constructed by deletion in adenovirus genome E1 region of about 4 Kb (sometimes also including partial deletion in E3 region of 3.6 Kb). This vector is most widely used today. The second generation Ad vectors are constructed on the foundation of first generation Ad vectors for further deletions of E2 and E4 genes to weaken its immunogenicity. But the second generation Ad vectors are less used today. The third generation Ad vectors are called the gutless Ad (GL-Adv) vectors, which have the deletions of all adenovirus genome's code regions, only retain its reverse terminal repetition sequence (ITR) and the viral packing signal (ψ). Foreign genes inserted in GL-Adv vectors can be expressed for a long time because it has no immunogenicity and can not be eliminated by the antibody.

The adeno-associated virus (AAV) is a single chain DNA virus with very low immunogenicity. It may insert to the chromosome at the fixed point, and no any poisonous action or the carcinogenicity is discovered at present uses. The retrovirus (RTV), lentivirus, HSV and the EB virus are also the more commonly used viral vectors.

The vectors for gene therapy carry the gene to the goal place for its function (for example anti-cancer function). The vector is a key for the gene therapy, and another essential factor is gene. The genes related to tumor therapy gene may be the tumor suppressor gene, cytokine gene and so on. IL-24 is a new cytokine, and has a good anti-cancer efficacy by suppressing cell growth and inducing apoptosis specifically in cancer cell. IL-24 can activate the immune system to kill the cancer cells. Also, IL-24 has antiangiogenic effects by enhancing the differentiation of vascular endothelial cells in cancer tissues to suppress the tumor growth. The above antitumor effect of IL-24 suggests its promising value for cancer gene therapy (Su, Z. Z. et al. Oncogene, 22, 1164-1180, 2003; Leath, C A et al. Gynecol. Oncol., 94,352-362, 2004; Lebedeva, I. V. et al. Oncogene, 21,708-718, 2002).

Several hundred protocols of gene therapy have been established, but there has been no clinical breakthrough so far. Therefore, viral therapy for cancer is becoming increasingly popular again. Among them, the recombinant ad which the ONYX medicine company develops (ONYX-015, also calls dl1520) is deleted the adenovirus genome's E1B55K protein gene, enables this recombinant ad to replicate selectively in tumor cell with the p53 dysfunction, but cannot replicate in the normal cell. The combination of ONYX-015 and chemotherapy (5-FU and cisplatin) achieved a therapeutic effect of more than 60%. When using ONYX-015 alone, the efficacy is less than 15%. So at 2000, professor Liu proposed a new strategy called “targeting gene-virotherapy”, which combines the advantages of gene therapy and viral therapy (Chinese J. Cancer Biother, 8 (1): 1). First we construct recombinant ad vector ZD55 with the deletion of E1B55K protein gene, which is similar to the ONYX-015 replicating selectively in tumor cell with the p53 dysfunction, but is a recombinant directly from Ad5 (by 2268 bp-3328 bp deletion of wild type Ad5) and has a clone site for the insertion of foreign gene. Different anti-cancer genes can be inserted into ZD55 to form ZD55-gene easily. Anti-cancer therapy by ZD55-gene is called Gene-ViroTherapy.

### SUMMARY OF THE PRESENT INVENTION

An object of the present invention is to provide a novel tumor-targeting recombinant gene-virus ZD55-IL-24, which can express IL-24 specifically in cancer cells with high efficiency by combining the advantages both of gene therapy and virotherapy.

Another object of the present invention is to provide the construction methods of the novel recombinant tumor-targeting gene-virus, ZD55-IL-24.

Another object of the present invention is to provide the application of the novel recombinant tumor-targeting gene-virus ZD55-IL-24, for cancer therapy.

Accordingly, in order to accomplish the above object, the present invention provides a recombinant tumor-targeting gene-virus, ZD55-IL-24. It is a recombinant Ad5 with the deletion of E1B 55 Kda gene carrying the anti-cancer gene IL-24.

This present invention provides a construction method of the novel recombinant tumor-targeting gene-virus, ZD55-IL-24 including the following steps.

A: The nested PCR uses pXC1 as the template were performed to construct plasmid pZD55 by deleting the E1B55 region (2269-3327 bp) of wild type Ad5.

B: The anti-cancer gene, IL-24, was cloned into pCA13, then the expression cassette of IL-24 was cut out by restriction endonucleases Bgl□, which contains CMV promoter, the anti-cancer geneIL-24, and SV40 poly A, and inserted into the tumor-targeting replicated adenoviral vector pZD55 to construct plasmid pZD55-IL-24.

C: Co-transfect plasmid pZD55-IL-24 with the adenovirus packaging plasmid pBHGE3 into the 293 cell to generate the recombinant gene-virus ZD55-IL-24, by standard homologous recombination.

The construction methods of the novel recombinant gene-virus ZD55-IL-24, in this invention may be used in developing the new effective tumor therapy medicine.

This novel recombinant gene-virus ZD55-IL-24 has the following beneficial effect:

1. This invention provides the novel recombinant gene-virus, ZD55-IL-24. In vitro cell experiments showed that anti-cancer gene IL-24 only selectively highly expressed in tumor cell but not or very low in the normal cell. In vitro cell experiments and in vivo the animal experiments showed that ZD55-IL-24 can be used in the therapy of many kinds of tumors.

2. This invention provides the construction methods of the novel recombinant gene-virus, ZD55-IL-24. These methods are easy to grasp.

3. The novel recombinant gene-virus, ZD55-IL-24 is the tumor-targeting replication ad which can replicate and express its carried anti-cancer gene IL-24 selectively in the tumor cells. Therefore ZD55-IL-24 has the very high targeting antitumor effect.

4. In vivo animal model experiments indicated that ZD55-IL-24 in this invention can kill the tumor cells selectively, but not the normal cells. Anti-tumor protein IL-24 expressed by ZD55-IL-24 enhances the antitumor effect of virus. The tumor in animal model in vivo can be almost completely eliminated by ZD55-IL-24. It will build the good foundation for clinic tumor therapy from now on.

### BRIEF DESCRIPTION OF THE DRAWINGS

FIG. 1 is a perspective view of . . . according to a preferred embodiment of the present invention.

FIG. 1A is a schematic drawing of construction for plasmid pZD55-IL-24.

FIG. 1B is a schematic overview of construction procedure for the recombinant gene-virus, ZD55-IL-24.

FIG. 2 shows the replication ability of the tumor-targeting recombinant gene-virus, ZD55-IL-24 in tumor cells and normal cells, illustrating that ZD55-IL-24 can selectively replicate in tumor cells.

FIG. 3 is IL-24 expression of ZD55-IL-24 in colorectal cancer cells and normal cells.

FIG. 4A to 4C shows cell viability of tumor cells (4A, 4B) and normal cells (4C) after 3 days post-infection with ZD55-IL-24 at 10 MOIs by MTT assay.

FIG. 5 shows ZD55-IL-24 selectively induced apoptosis in colorectal cancer cells with Hochest staining.

FIG. 6 illustrates antitumor activity of tumor-targeting recombinant gene-virus ZD55-IL-24 on SW620 xenograft model.

### DETAILED DESCRIPTION OF THE PREFERRED EMBODIMENT

This invention is further explained with the concrete examples hereinafter. Should be understood, the following examples are only used in explaining this invention but not in defining the scope of this invention.

The tumor-targeting recombinant gene-virus ZD55-IL-24 of this invention was deposited in CHINA CENTER FOR TYPE CULTURE COLLECTION (CCTCC) at May 20, 2005. The name is recombinant tumor-targeting adenovirus expressing IL-24: ZD55-IL-24. The number of deposition is CCTCC-V200505.

### Example I

### Construction of Tumor-Targeting Gene-Virus Zd55-IL-24, which Carries the Anti-Cancer Gene IL-24

A. The Construction of Tumor-Targeting Plasmid pZD55

First, design the following primers:

Xba□A primer:
5′ GCC GAC ATC ACC TGT G TCT AGA GAA TG 3′;
Xba IB primer:
5′ TCA GAT GGG TTT CTT CAC TCC ATT TAT CCT 3′;
Bgl IIA primer:
5′ ATA AAG GAT AAA TGG AGT GAA GAA ACC CAT
CTG AG 3′;

(The third codon of 55 KDa gene is changed to stop codon, C2024T)

Bgl IIB primer:
5′ GA AGA TCT ATA CAG TTA AGC CAC CTA TAC AAC
A 3′;

(The open reading frame (ORF) of E1B the 55 KDa gene is changed and two stop codons are inserted, C2252T, G2261T)

Plasmid pXC1 (from Microbix Biosystem Inc, Toronto) contains Ad5 sequences from 22 bp-5790 bp (0-16.1 mu).

The viral region comprising 719 bp nucleotides was obtained by polymerase chain reaction (PCR) (for details please see Molecular Cloning: A Laboratory Manual, 3rd ed., Joseph Sambrook and David W. Russell) with pXC1 as the template using Xba IA primer and Xba TB primer. This PCR product is called Z1. In a similar way, another 270 bp PCR product called Z2 is obtained by PCR with pXC1 as the template using Bgl IIA primer primer and Bgl IIB primer. Both PCR products contain a 34-bp random sequence at the 5′ end, which is complementary to each other.

Using the mixture of Z1 and Z2 as template, a fragment of about 955 bp was amplified using the primers of Xba IA and Bgl IIB. This The PCR product is called Z3. Z3 is digested with XbaI/Bgl II and ligated into XbaI/Bgl II sites of pXC1 to construct the plasmid pXC1-D55.

The plasmid pCA13 (from Microbix Biosystem Inc, Toronto) contains the SV40 poly A. This 160 bp SV40 poly A was obtained by digesting the pCA13 with BamHI/Bgl II and ligated into Bgl II sites of pXC1-D55. After the appraisal with restriction endonucleases, the positive-direction cloning plasmid is named pZD55. pZD55 includes the deletion of EIB 55 KDa gene from 2268 bp to 3328 bp.

B. The Construction of Tumor-Targeting Plasmid pZD55-IL-24

First, designs the following primers:

| | |
|---|---|
| IL-24 A: | 5′GTACTCGAGATGAATTTTCAACAGAGGCTGC3′; |
| Xho I | |
| IL-24 B: | 5′ ATGGATCCTCAGAGCTTGTAGAATTTCTGC 3′; |
| BamH I | |

There are many MCS like Sal I, Hind III, EcoR I, EcoR V, Xba I, Xho I, BamH I between the CMV promoter and SV40 poly A in plasmid pCA13. By gene manipulation, the positive-direction insertion of anti-tumor gene into plasmid pCA13 at MCS formed plasmid pCA13-gene (for details, please see Molecular Cloning: A Laboratory Manual, 3rd ed., Joseph Sambrook and David W. Russell). The expression cassette of anti-tumor gene can be cut out by Bgl□ which includes CMV promoter, anti-tumor gene and SV40 poly A. Then this expression cassette of anti-tumor gene was cloned into Bgl□ digested and dephosphorylated pZD55 to construct plasmid pZD55-gene.

The detailed construction procedures of pZD55-IL-24 are as following:

Using primers of IL-24 A and IL-24 B, the 641 bp PCR product containing IL-24 gene was obtained by PCR reaction using the plasmid pCDNA3-IL-24 (from Wuhan three hawks biological technology company, Wuhan) as the PCR reaction template. This PCR product was cut by Xho I/BamH I, then cloned into Xho I/BamH I digested pCA13 to form the plasmid pCA13-IL-24.

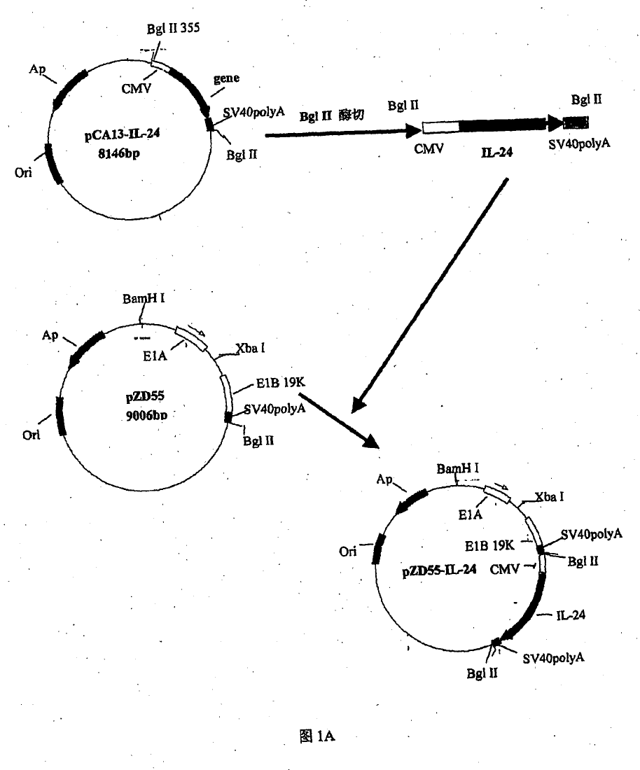

The expression cassette of IL-24 gene can be cut out by Bgl□ from plasmid pCA13-IL-24, which includes CMV promoter, IL-24 gene, and SV40 poly A. Then this expression cassette of IL-24 gene was cloned into Bgl□ digested and dephosphorylated pZD55 to construct plasmid pZD55-IL-24. The schematic drawing of construction for plasmid pZD55-IL-24 is showed in FIG. 1A. The correct construction of pZD55-IL-24 was confirmed by DNA sequencing.

C. The Construction of Tumor-Targeting Gene-Virus ZD55-IL-24

Plasmid pBHGE3 and 293 cells are from Microbix Biosystem Inc. (Toronto, Canadian). Plasmid pBHGE3 includes the Ad5 sequences with a deletion of Ad5 E1 sequences from 188 bp to 1339 bp. 293 cells (Microbix Biosystem Inc., Toronto, Canadian) are human embryo kidney cells that transformed by sheared adenovirus type 5 DNA. They contain and express the E1 region of ad5 and have high transfection efficacy with adenovirus DNA.

Plasmid pZD55-IL-24 (with Ad5 left arm sequences of homologous recombination) is cotransfected 293 cells with plasmid pBHGE3 (with Ad5 skeleton DNA sequences) to produce recombinant adenovirus ZD55-IL-24 (FIG. 1B). The detailed recombinant method is according to the handbook of Qiagen Corporation. About 7-14 days after transfection, the individual plagues emerged in HEK293 cells. After plague purification two times, amplify the recombinant adenovirus, extract the ad DNA and the ad DNA is analyzed by restriction endonucleases digestion and PCR to determine the correct recombinant adenovirus ZD55-IL-24.

The detailed procedures of recombinant ad plague purification, amplification

and appraisal: 293 cells are plated on 6-well tissue culture plates and incubated 24 h at 37° C. Near confluence cells are infected with different diluted viral stocks (such as 10-6, 10-7, 10-8, 10-9). After 2 h post-infection, proceed to overlaying the infected cells with 3 ml low melting point agarose (10% FBS, 1.25% Agarose). About 9 days after transfection, the individual plagues emerged in HEK293 cells. Core-out well isolated plaques and transfer to 250 μl of growth medium in a sterile microcentrifuge tube, then add to the 24-well tissue culture plates with near confluence 293 cells. Viral DNA is obtained with Qiagen Blood Kit. The appraisal of gene-virus ZD55-gene is by PCR. The primers are synthesized by Shanghai Sengong. (Note: the number at right flank of the primers' sequences indicates the sequence number which pairs sequences of the plasmid pXC1).

| | |
|---|---|
| ZD55 sense primer: | |
| 5′ AGA GCC CAT GGA ACC CGA GA 3′; | bp 2200-2219 |
| ZD55 antisense primer: | |
| 5′ CAT CGT ACC TCA GCA CCT TCC A 3′; | bp 3353-3332 |

Using viral DNA extracted by Qiagen Blood Kit as the template, PCR reactions are carried on with the ZD55 sense primer and ZD55 antisense primer, the wild viral DNA as a control. PCR condition: 94° C.×1 min, 55° C.×1 min, 72° C.×2 min 15 s. If the PCR product only contains gene, likely not contains 1113 bp wild adenovirus DNA, the plague purification is successful. Repeat this process to obtain the correct recombinant ad. The correct gene-virus ZD55-IL-24 was deposited in CHINA CENTER FOR TYPE CULTURE COLLECTION (CCTCC). The name is recombinant tumor-targeting adenovirus expressing IL-24: ZD55-IL-24. The number of deposition is CCTCC-V200505.

Adenovirus ZD55-IL-24 is largely amplified in 293 cells and purified by cesium chloride gradient. The detailed operating procedure is according to handbook of Microbix Biosystem Inc.

### Example II

### Replication Assay of Gene-Virus ZD55-IL-24 in Tumor or Normal Cell

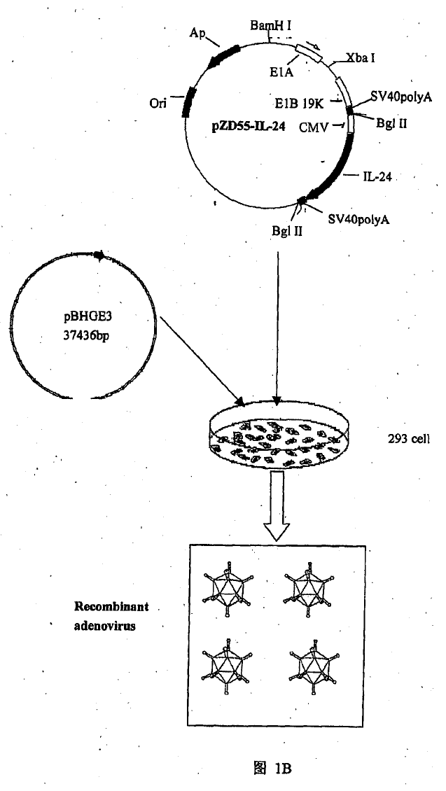

To determine virus progeny, 3×105 tumor or normal cells were plated in 6-well plate. After 24 h, human hepatocarcinoma cell line BEL7404 (from the Shanghai Cell Collection), the human breast carcinoma cell line Bcap37, the human cervical carcinoma cell line Hela, the human colorectal carcinoma cell line SW620, HT-29, HCT116, and normal human lung fibroblasts NHLF (from ATCC) are infected with 104 PFU ZD55-IL-24, Ad-IL-24 or ONYX-015 respectively. After 48 hr, medium and cells were collected, and virus was released by freeze-thawing for three cycles and centrifuged to collect the supernatant. Virus production was determined by standard plaque assay on 293 cells. Plate 293 cells in 60 mm dish and incubate 24 h at 37° C. When cells grow confluence, they are infected with different diluted viral stocks (such as 10-1, 10-2, 10-3, 10-4, 10-5, 10-6, 10-7, 10-8) in 1-ml volumes. 2 h post-infection, gently pipette 8 ml of agarose/growth medium (5% FBS, 1.25% Agarose) and allow it to completely cover the bottom of the dish. About 9 days later, count plaques from wells where isolated plaques are clearly visible and countable. Calculate viral progeny number which each PFU virus produced. Results are showed in FIG. 2.

The replication ability of ZD55-IL-24 or ONYX-015 in tumor cell increased significantly compared with Ad-IL-24. But in normal cell, the replication ability of ZD55-IL-24 or ONYX-015 remarkably decreased. There is no obvious difference between the replication ability of Ad-IL-24 in tumor cell and in normal cell. So Ad-IL-24 has not the selectivity. But ZD55-IL-24 or ONYX-015 can selectively replicate in the tumor cells.

### Example III

### Characterization of Gene-Virus ZD55-IL-24 Expressing IL-24 Protein in Normal Cell and Tumor Cells

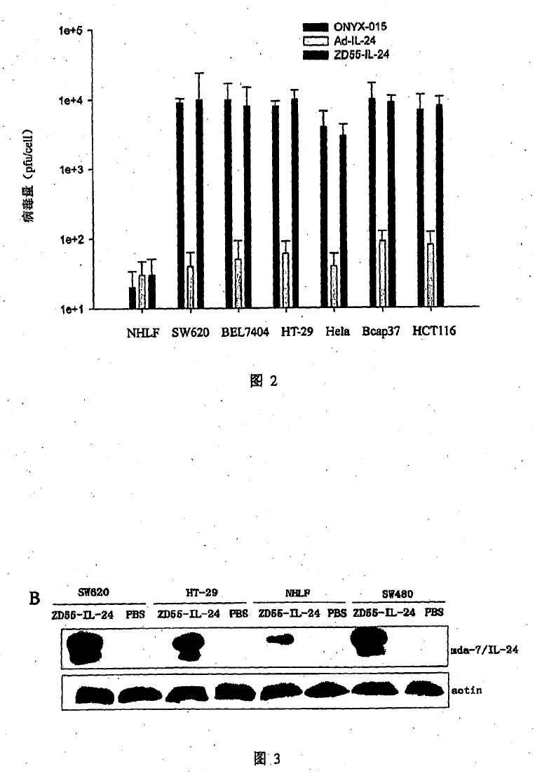

To examine exogenous IL-24 expression, 3×105 tumor or normal cells were plated in 6-well plate. After infection with ZD55-IL-24 at an MOI of 5 for 48 hr, colorectal cancer cells (SW620, HT-29, and SW480) and normal human lung fibroblasts (NHLF) were harvested and subjected to Western blot. As shown in FIG. 3, all three colorectal cell types infected with ZD55-IL-24 expressed much higher IL-24 protein compared with the control cell line. And NHLF cells infected with ZD55-IL-24 only show less IL-24 expression.

### Example IV

### The Killing Effect of Gene-Virus ZD55-IL-24 on Tumor Cell In Vitro

After cell is infected with virus, cell viability is determined by MTT assay (Cancer Research, 1989, 49(17):4785-90). Steps are as follows: plate colorectal carcinoma cell line SW620, HT-29 and normal human embryo lung cell NHLF in 96-well plate by 5,000 cell per well and incubated at 37° C. After 24 h cells are infected by 10 MOIs of virus. 3 days postinfection, the medium containing virus is removed and normal medium containing 5 mg/ml MTT is added. For further more 4 h incubation, the medium containing MTT is removed and solubilization solution is added to each well and mixed thoroughly for 4 h. Absorbance from the plates was read at 655 and 595 nm.

Cell viability (%)=A595(sample)/A595(control)×100%.

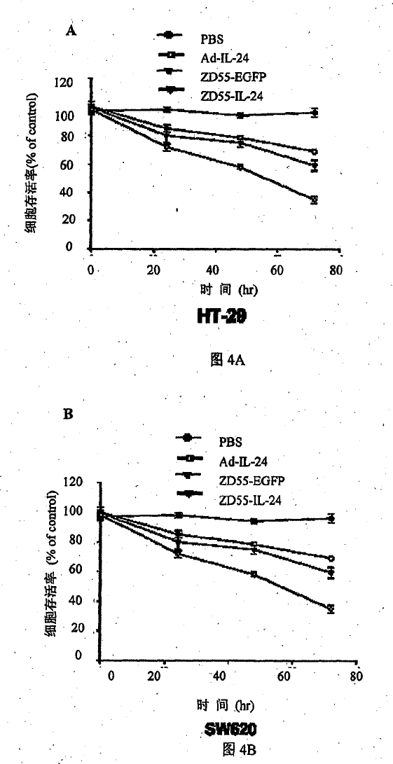

As indicated in FIGS. 4A, 4B and 4C, ZD55-IL-24 has very obviously killing effect on the tumor cells, but very low toxicity to the normal cell, so has the tumor selectivity.

### Example V

### The Gene-Virus ZD55-IL-24 Selectively Induces Apoptosis of Cancer Cells In Vitro

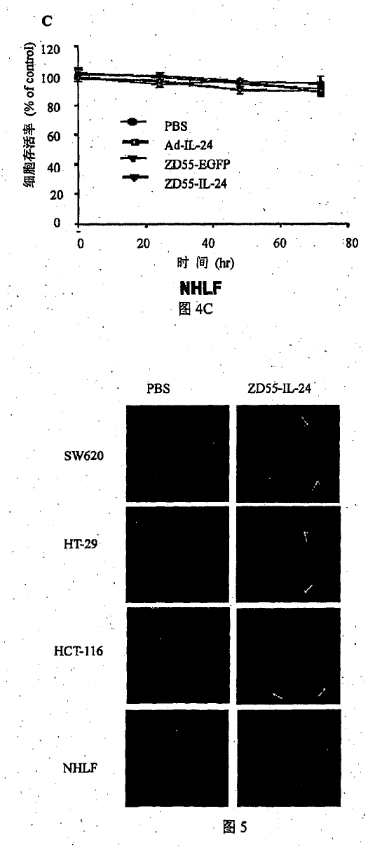

To determine virus progeny, 3×105 tumor cells (SW620, HT-29, HCT-116) or normal cell (NHLF) were plated in 6-well plate. After 24 h, cells are infected with 1 MOI ZD55-IL-24, and PBS as control. After 48 hr, cells were analyzed for apoptotic changes by Hoechst 33258 staining for 10 min. FIG. 5 indicates that tumor cells but not normal cells treated with ZD55-IL-24 showed obvious apoptosis and no changes were observed in any of the cells treated with PBS. Arrows indicate apoptotic cells.

### Example VI

### Antitumor Efficacy of ZD55-IL-24 on Xenograft in Nude Mice In Vivo

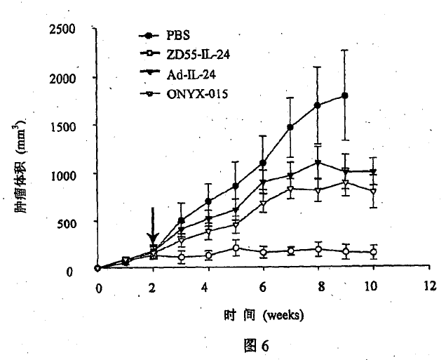

Subcutaneous tumors are established in the right flank of nude mice of 4-week old using human colorectal carcinoma cell line SW620. About 12 days later, when the tumors reached 100-150 mm3, they were treated with 1×109 pfu ZD55-IL-24 or Ad-IL-24 as experimental groups, PBS or 1×109 pfu ONYX-015 as control groups, by intratumoral injection. The results showed in FIG. 6 that the antitumor efficacy of ZD55-IL-24 is best in all groups. 9 weeks after treatment, the tumor is almost completely eliminated in ZD55-IL-24 group, and its antitumor effect is significantly better than that of ONYX-015 or Ad-IL-24.

What is claimed is:

1. A tumor-targeting gene-virus ZD55-IL-24, which is a recombinant Ad5 with a deletion of EIB 55 Kda gene and carries the anti-tumor gene IL-24.

2. The tumor-targeting gene-virus ZD55-IL-24 of claim 1, wherein the construction method includes following steps of:
A: The nested PCR using pXC1 as the template were performed to construct plasmid pZD55 by deleting the EIB 55 Kda region (2269-3327 bp) of wild type Ad5.
B: The anti-cancer gene IL-24 was cloned into pCA13, then the expression cassette of IL-24 was cut out by restriction endonucleases Bgl □, which contains CMV promoter, the anti-cancer gene IL-24, and SV40 poly A, and inserted into the tumor-targeting replicated adenoviral vector pZD55 to construct plasmid pZD55-IL-24.
C: Co-transfect plasmid pZD55-IL-24 with the adenovirus packaging plasmid pBHGE3 into the 293 cell to generate the recombinant gene-virus ZD55-IL-24, by standard homologous recombination.

3. The construction method of claim 2, wherein the expression cassette includes CMV promoter, the anti-cancer gene IL-24, and SV40 poly A at step B.

4. The construction method of claim 2, wherein the adenovirus packaging plasmid is pBHGE3 at step C.

5. The tumor-targeting gene-virus ZD55-IL-24 of claim 1, wherein it can be used in the therapy of many kinds of tumors.
